(19) United States
(12) Patent Application Publication
(10) Pub. No.: US 2023/0238079 A1
(43) Pub. Date: Jul. 27, 2023

(54) METHOD, KIT AND COMPUTER-IMPLEMENTED METHOD FOR PREDICTING SURVIVAL TIME OF INDIVIDUAL WITH BLADDER CANCER AFTER SURGERY FROM INDIVIDUAL'S BIOLOGICAL SAMPLE

(71) Applicant: National Sun Yat-Sen University, Kaohsiung (TW)

(72) Inventors: Jim Jinn-Chyuan Sheu, Kaohsiung (TW); Chung Chang, Kaohsiung (TW); Bo-Chen Lin, Kaohsiung (TW); Li-Yun Yang, Kaohsiung (TW); You-Jun Lai, Kaohsiung (TW)

(73) Assignee: National Sun Yat-Sen University, Kaohsiung (TW)

(21) Appl. No.: 18/146,571

(22) Filed: Dec. 27, 2022

(30) Foreign Application Priority Data
Jan. 21, 2022 (TW) 111102676

(51) Int. Cl. G16B 20/00; G16B 40/20; G16H 50/30

(52) U.S. Cl. CPC G16B 20/00; G16B 40/20; G16H 50/30

(57) ABSTRACT

The present invention relates to a method and a kit, a computer-implemented method and a system for in vitro predicting survival time of an individual with bladder cancer after surgery from an individual's biological sample. Expression levels of a target gene combination of in vitro aggressive bladder cancer specimen of a patient are detected, and the target gene combination includes at least two of PPT2, ARMH4, P4HB, SLC1A6 and ARID3A, a fragment, a homologue, a variant and a derivative thereof. Next, the expression levels are respectively compared with the reference expression levels of a reference database, and converted to a risk score sum, thereby predicting an averaged survival time of a patient having aggressive bladder cancer after surgery, and being beneficially applied to a kit and a computer-implemented method for in vitro predicting survival time of patient with most aggressive types of bladder cancer after surgery.

### RELATED APPLICATIONS

This application claims benefit of priority from Taiwan Application No. 111102676, filed on Jan. 21, 2022. The entire disclosures of all the above applications are hereby incorporated by reference herein.

### BACKGROUND

### Field of Invention

The present invention relates to a method and application for in vitro predicting survival time of individual with bladder cancer after surgery. More specifically, the present invention relates to a method, kit, and a computer-implemented method for predicting survival time of individual with bladder cancer after surgery from an individual's biological sample according to expression levels of specific target gene combination as criteria to molecular typing.

### Description of Related Art

Bladder cancer is a common aggressive disease of the urinary system. Though bladder cancer can occur at any age, its risk rises with age, especially in the age group of 50 to 70 years old. Bladder cancer is three to four times more frequent in men than women. In Taiwan, bladder cancer is not among the top ten cancer deaths (men and women combined), but bladder cancer ranked 9th in male cancers in Taiwan in 2017. Thus, bladder cancer is one of the common urological malignancies.

The known risk factors of bladder cancer include smoking, race, genetics, chronic bladder inflammation in a long term, receiving anti-cancer medication, and the environment (such as those working in rubber, chemical dyes, printing, leather shoes, hair dye, paint, etc.).

According to the depth of tumor invasion, bladder cancer can be classified into non-muscle invasive bladder cancer (NMIBC) and muscle invasive bladder cancer (MIBC). Approximately 75% of newly diagnosed bladder cancers are NMIBC, and MIBC accounts for approximately 25% of bladder cancers. The survival rate of NMIBC patients is relatively high, but approximately 60% of the NMIBC patients develop recurrence even after surgery, and approximately 80% of the NMIBC patients develop recurrence within one year after surgery, while approximately 15% progress to MIBC. Once diagnosed with metastatic or advanced unresectable bladder cancer, approximately 70% of patients will die within 2 years. The 5-year survival rate of those patients is about 60% with radical surgery, but the 5-year survival rate of the patients with lymph node metastasis is only approximately 15%, and the 5-year survival rate of patients with other organ metastasis is approximately 5%.

Arriving at a diagnosis and a treatment of bladder cancer may involve intravenous pyelography (IVP) and cystoscope, both of which are often used to detect bladder cancer and to assist the surgery (e.g., transurethral resection of bladder tumor, TUR-BT). Chemotherapy and radiation therapy can be optionally combined after surgery. In general, bladder cancer can be treated successfully when detected at an early stage, and the average 5-year survival rate after surgery can reach approximately 60%. However, when the bladder is assessed by IVP using a cystoscope, there are problems such as dead zones of the lens and blurry images sometimes difficult to ascertain, leading in a high recurrence rate after surgery. Periodic follow-up observations have to be organized, and TUR-BT can be repeated if necessary during the follow-up period. However, before, during and after the TUR-BT operation, various pains, inconvenience and discomfort make the patients feel stressed, be less willing to undergo surgery, even ignore the importance of regular check-ups, resulting in possible deterioration and metastasis of cancers.

The bladder cancer is a highly heterogeneous disease in the molecular genetics and histopathology. Most NMIBC carcinomas are grown as papillary shapes, while MIBC carcinomas are associated with flat atypical hyperplasia and carcinoma in situ. Fibroblast growth factor receptor 3 (FGFR3) promoter mutations are frequent in NMIBC. Inactivation or mutation of p53 gene may confer MIBC with highly genetic instability.

Since the bladder cancer is a highly heterogeneous disease, the conventional histopathological classification cannot meet the clinical requirement. There is an urgent need to discover molecular typing markers of the bladder cancer for predicting the survival time of an individual with the bladder cancer after surgery, thereby assisting to choose a more suitable treatment strategy.

### SUMMARY

In an aspect, the invention provides a method for in vitro predicting survival time of an individual with bladder cancer after surgery from an individual's biological sample. In the method, expression levels of target gene combination of in vitro aggressive bladder cancer specimen of an individual are detected, in which the target gene combination can include at least two of PPT2, ARMH4, P4HB, SLC1A6 and ARID3A, a fragment, a homologue, a variant and a derivative thereof. The expression levels of the target gene combination are respectively compared with the reference expression levels of the target gene combination of a reference database, and converted to a risk score sum. When the risk score sum of an individual is equal to or greater than a threshold, the individual is classified into a high risk group, thereby increasing the predicting accuracy of an averaged survival time of an individual having aggressive bladder cancer after surgery.

In yet a further aspect, the invention also provides a computer-implemented method for in vitro predicting survival time of an individual with bladder cancer after surgery from an individual's biological sample. The computer-implemented method includes the steps of providing a biological specimen, detecting expression levels of the target gene combination of the biological specimen, comparing one of the expression levels with a corresponding reference expression level of the target gene combination of a reference database respectively, obtaining a difference and a risk score, and calculating a risk score sum of at least two of the target gene combination of the biological specimen, in which the step of comparing one of the expression levels with a corresponding reference expression level of the target gene combination of a reference database respectively, the step of obtaining a difference and a risk score, and the step of calculating a risk score sum of at least two of the target gene combination of the biological specimen can be executed within a computer system for implementing the aforementioned method.

In yet a further aspect, the invention also provides a system for in vitro predicting survival time of an individual with bladder cancer after surgery from an individual's biological sample. The system includes a detecting module, a comparing module, a determining module and a controlling system, thereby increasing the predicting accuracy of an averaged survival time of an individual having aggressive bladder cancer after surgery.

According to the aforementioned aspect, the present invention provides a method for in vitro predicting survival time of an individual with bladder cancer after surgery from an individual's biological sample. In an embodiment, the method can include the step of establishing a reference database. Next, a biological sample is provided. And then, expression levels of the target gene combination of the biological specimen are detected, in which the target gene combination can include at least two of PPT2, ARMH4, P4HB, SLC1A6 and ARID3A, a fragment, a homologue, a variant and a derivative thereof. Later, one of the expression levels with a corresponding reference expression level of the target gene combination of a reference database respectively, for obtaining a difference and a risk score. A risk score of the one is given as 1 when an absolute value of the difference is equal to or more than a first threshold that is more than at least 5% of the reference expression level. Consequently, a risk score sum of at least two of the target gene combination of the biological specimen is calculated. When the risk score sum is equal to or greater than a second threshold that is 1 or 2, the individual having the biological specimen is classified to a high risk group, wherein the high risk group is defined by less 25 months of an averaged survival time after surgery of the individual with 1 or 2 of the second threshold, or by less 10 months of an averaged survival time after surgery of the individual with 3 of the second threshold.

According to another aspect, the invention provides a kit for in vitro predicting survival time of an individual with bladder cancer after surgery from an individual's biological sample. The kit includes a reaction solution, nucleic acid probes and/or antibodies, in which the nucleic acid probes and/or antibodies react with a target gene combination of a biological specimen and generate expression levels, the biological specimen is originated from in vitro aggressive bladder cancer of an individual, and the target gene combination is selected from at least two of the group consisting of PPT2, ARMH4, P4HB, SLC1A6 and ARID3A, a fragment, a homologue, a variant and a derivative thereof.

According to yet another aspect, the invention provides a computer-implemented method for in vitro predicting survival time of an individual with bladder cancer after surgery from an individual's biological sample. In an embodiment, the computer-implemented method includes the steps of storing a reference data bank in a system, detecting expression levels of the target gene combination of a biological specimen, comparing one of the expression levels with a corresponding reference expression level of the target gene combination, calculating a risk score sum of at least two of the target gene combination, and identifying the individual from the risk score sum. A program executes the aforementioned steps of storing, comparing, calculating and identifying, for implementing the method on a computer system.

According to still another aspect, the invention provides a system for in vitro predicting survival time of an individual with bladder cancer after surgery from an individual's biological sample. In an embodiment, the system includes a detecting module, a processing module, an identifying module and a controlling module.

In the aforementioned embodiment, the detecting module can include a detecting device, a reaction solution, nucleic acid probes and/or antibodies. The nucleic acid probes and/or antibodies react with a target gene combination of a biological specimen and generate expression levels, and the detecting device can detect the expression levels. The biological specimen can be originated from in vitro aggressive bladder cancer of an individual. The target gene combination can include but be not limited to at least two of the group consisting of PPT2, ARMH4, P4HB, SLC1A6 and ARID3A, a fragment, a homologue, a variant and a derivative thereof.

The processing module can be coupled to the detecting module, for receiving the expression levels from the detecting module and comparing one of the expression levels with a corresponding reference expression level of the target gene combination of a reference database respectively, thereby obtaining a difference and a risk score; and for calculating a risk score sum of at least two of the target gene combination of the biological specimen. The risk score of the one is given as 1 when an absolute value of the difference is equal to or more than a first threshold that is more than at least 5% of the reference expression level.

The identifying module can be coupled to the processing module, for identifying the individual from the risk score sum. When the risk score sum is equal to or greater than a second threshold that is 1 or 2, the individual having the biological specimen is classified to a high risk group, in which the high risk group is defined by less 25 months of an averaged survival time after surgery of the individual with 1 or 2 of the second threshold, or by less 10 months of an averaged survival time after surgery of the individual with 3 of the second threshold.

The controlling module can be coupled to the detecting module, the processing module and the identifying module. The system executes a program that includes instructions for activating the detecting module, the processing module and the identifying module.

In the aforementioned embodiments, the computer system further optionally includes a pretreating module coupling to the detecting module, for providing a nucleic acid sample and/or a protein sample of the biological specimen.

With application to the method and the kit for in vitro predicting survival time of an individual with bladder cancer after surgery from an individual's biological sample, which detect expression levels of target gene combination of in vitro aggressive bladder cancer specimen of an individual, respectively compare those expression levels with the reference expression levels of the target gene combination of a reference database, the results can be converted to a risk score sum, thereby increasing the predicting accuracy of an averaged survival time of an individual having aggressive bladder cancer after surgery, leading in applications on the computer-implemented method and the system for in vitro predicting survival time of an individual with bladder cancer after surgery from an individual's biological sample.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in color. Copies of this patent or patent application publication with color drawings will be provided by Office upon request and payment of the necessary fee. The disclosure can be more fully understood by reading the following detailed description of the embodiment, with reference made to the accompanying drawings as follows.

FIG. 1A shows the computer-implemented method for in vitro predicting survival time of an individual with bladder cancer after surgery from an individual's biological sample according to an embodiment of the present invention, which involves a univariate analysis result of the bladder cancer patient database using Cox regression analysis and FDR correction.

FIG. 1B shows the computer-implemented method for in vitro predicting survival time of an individual with bladder cancer after surgery from an individual's biological sample according to an embodiment of the present invention, which involves a multivariate and high-dimensional analysis result of genes in FIG. 1A using the Lasso algorithm (the left panel of FIG. 1B) and the adaptive Lasso algorithm (the right panel of FIG. 1B).

FIG. 2A shows 26 aggressive bladder cancer-related genes screened from TCGA gene database according to an embodiment of the present invention, which uses the right panel of FIG. 1B involving the computer-implemented method for in vitro predicting survival time of an individual with bladder cancer after surgery from an individual's biological sample.

FIG. 2B shows a scatter plot representing univariate and multivariate predicted risk coefficients of the 26 genes screened from FIG. 2A.

FIG. 3A shows the 26 genes screened from FIG. 2A according to an embodiment of the present invention.

FIG. 3B shows a matrix diagram of correlation analysis of known activating mutations in the oncogene list according to an embodiment of the present invention.

FIG. 4 shows a heat map representing genetic risk coefficients of MIBC samples of GEO database (GSE13507) (the right panel) and TCGA database (the left panel) according to an embodiment of the present invention.

FIG. 5 shows a ranking heat map of the significance [log (p value)] of gene expression levels of MIBC samples of GEO database (GSE13507)(the right panel) and TCGA database (the left panel) according to an embodiment of the present invention, both of which are analyzed by Cox proportional-hazards model (CoxPH Model).

FIGS. 6A to 6B respectively show Kaplan-Meier survival curves and survival rates of genes screened from TCGA database (FIG. 6A) and GEO database (FIG. 6B) according to an embodiment of the present invention.

FIGS. 7A to 7C respectively show several gene enrichment plots related to ARID3A overexpression according to the computer-implemented method for in vitro predicting survival time of an individual with bladder cancer after surgery from an individual's biological sample of the present invention.

FIGS. 8A to 8D respectively show several gene enrichment plots related to ARMH4 overexpression according to the computer-implemented method for in vitro predicting survival time of an individual with bladder cancer after surgery from an individual's biological sample of the present invention.

FIGS. 9A to 9D respectively show several gene enrichment plots related to P4HB overexpression according to the computer-implemented method for in vitro predicting survival time of an individual with bladder cancer after surgery from an individual's biological sample of the present invention.

FIGS. 10A to 10C respectively show several gene enrichment plots related to PPT2 overexpression according to the computer-implemented method for in vitro predicting survival time of an individual with bladder cancer after surgery from an individual's biological sample of the present invention.

FIGS. 11A to 11E respectively show several gene enrichment plots related to SLC1A6 overexpression according to the computer-implemented method for in vitro predicting survival time of an individual with bladder cancer after surgery from an individual's biological sample of the present invention.

FIG. 12 shows various metabolic pathways associated with aggressive bladder cancer by combining 5 target genes according to several embodiments of the present invention.

FIGS. 13A to 13D shows histochemical staining images of normal bladder tissue sections (FIGS. 13A to 13B) and aggressive bladder cancer tissue sections (FIGS. 13C to 13D) with 4-fold magnification according to several embodiments of the present invention.

FIGS. 13E to 13X shows histochemical staining images of normal bladder tissue sections and aggressive bladder cancer tissue sections with 4-fold magnification according to several embodiments of the present invention.

### DETAILED DESCRIPTION

Where a definition or use of a term in an incorporated reference is inconsistent or contrary to the definition of that term provided herein, the definition of that term provided herein applies and the definition of that term in the reference does not apply. Moreover, unless the context otherwise requires, singular terms can include the plural and vice versa.

As aforementioned, the present invention provides a method for in vitro predicting survival time of an individual with bladder cancer after surgery from an individual's biological sample, in which expression levels of target gene combination of in vitro aggressive bladder cancer specimen of a patient are detected and compared with the reference expression levels of the target gene combination of a reference database, thereby predicting an averaged survival time of a patient having aggressive bladder cancer after surgery.

The terms “subject”, “individual” and “patient” are used interchangeably herein and refer generally to a subject with bladder cancer, and the subject can be a human or a non-human mammal. In an embodiment, the subjects with bladder cancer include NMIBC and MIBC individuals.

The term “survival time after surgery of the bladder cancer” generally refers to an averaged survival time after surgery of the individual with the bladder cancer, and it is also called as an averaged survival time after surgery of the bladder cancer. The “after surgery” typically refers to an individual with the bladder cancer after a radical surgery (e.g., radical cystectomy or partial cystectomy) or transurethral resection of bladder tumor (TUR-BT).

The term “biological sample” described herein can be originated from an in vitro aggressive bladder cancer from an individual. The reference expression level can be originated from an in vitro healthy bladder. The biological sample and the in vitro healthy bladder sample can include but be not limited to ex vivo organs, tissues, cells, body fluid, lymphatic liquid, urine, whole bloods, serum and/or cell culture supernatant, as well as nucleic acid extract (such as genomic DNA extract, mRNA extract, cDNA or cRNA obtained from the mRNA extract, etc.) or protein extract obtained from the aforementioned biological sample.

The term “target gene” or “target gene combination” described herein can include but be not limited to at least one of PPT2, ARMH4, P4HB, SLC1A6 and ARID3A, and the fragment, the homologue gene, the variant gene or the derivative gene of the aforementioned genes. In other examples, the target gene can be a biomarker.

The term “expression levels of target genes” can include expression levels of nucleic acids (DNA or RNA) and/or proteins. In some examples, one of the expression levels of target genes can be obtained as follows. A biological sample can be optionally pretreated to obtain nucleic acid extract and/or protein extract. Next, an expression level of one gene of the target gene combination (e.g., nucleic acid expression level and/or protein expression level) can be detected. In an example, the relative expression level of the gene can be normalized by a reference expression level (as a normalization data) of the target gene combination in a reference database corresponding to the gene from the nucleic acid extract and/or protein extract. In the aforementioned example, the reference database can include a plurality of expression levels of the target gene combination originated from at least one in vitro normal bladder sample.

The term “standardization” or “normalization” herein refers to standardizing or normalizing the data detected from the biological sample according to a standard the data from a normal (or healthy) sample, for further comparing and analyzing the data.

The term “risk score” herein refers to a difference obtained by comparing one of the aforementioned expression levels with the reference expression levels of the target gene combination in the reference database respectively, in which the absolute value of the difference that is equal to or greater than the reference expression level reaches a first threshold, a risk score of the one is given as 1. In this embodiment, there is no specific limitation to the first threshold, for example, at least 5%. In some embodiments, the expression levels of P4HB, SLC1A6 and ARID3A in the cancer lesion can be significantly greater than the ones in the normal tissue, so that the three biomarkers can be potentially applied to predict the averaged survival time after surgery of an individual with the bladder cancer in future. In other embodiments, the expression levels of PPT2 and ARMH4 in the surrounding tissue of the cancer lesion can be significantly higher than the ones in the normal tissue, so that the two biomarkers can be potentially as the biomarkers for predicting the precancerous lesion in future.

It should be clarified that, the difference, the first threshold or the risk score as aforementioned is merely illustrated but is not limited within the specific ranges or specific values. In other examples, the first threshold can be given to at least 6% to at least 10% or other values depending upon the actual requirements.

In an embodiment, a risk score sum of at least two of the target gene combination of the biological specimen can be calculated. When the risk score sum is equal to or more than a second threshold that is 1 or 2, the individual having the biological specimen is classified to a high risk group. In this embodiment, there is no specific requirement to the second threshold depending upon the definition of the risk score. For example, when the risk score is 1, the second threshold can be exemplified as 2; in some example, when the risk score is 0.5, the second threshold can be exemplified as 1; in certain examples, when the risk score is 2, the second threshold can be exemplified as 4. In the aforementioned embodiment, the “high risk group” is defined herein by less 25 months of an averaged survival time after surgery of the individual with 1 or 2 of the second threshold. In other embodiments, the “high risk group” is defined by less 10 months of an averaged survival time after surgery of the individual with 3 of the second threshold.

In an embodiment, a combination of other treatment can be optionally subjected to an individual after surgery. The term “treatment” can include but be not limited to chemotherapy and/or radiotherapy and/or surgery, for healing or improving the cancer or extending a lifespan of an individual. It should be supplemented that, the five target genes (or biomarkers) of PPT2, ARMH4, P4HB, SLC1A6 and ARID3A, as well as their upstream and downstream gene modulating pathways, can be as molecular typing markers, for designing personalized precision medicine, thereby elevating the diagnostic accuracy and treating effects.

It should be mentioned that, in the method for in vitro predicting survival time of an individual with bladder cancer after surgery from an individual's biological sample, the specific target gene combinations are used in the aforementioned method according to specific criteria (i.e., the difference, the risk scores, the first threshold, the second threshold), so as to accurately predict the survival time of an individual with bladder cancer after surgery. If other genes excepted from the specific target gene combinations were used in the aforementioned method or the criteria changed, such result according to the modified method could not accurately predict the survival time of an individual with bladder cancer after surgery.

In some embodiments, a computer-implemented method for in vitro predicting survival time of an individual with bladder cancer after surgery from an individual's biological sample can be applied to a computer program within a computer system for implementing the method, in which the computer-implemented method can include steps as follows. Firstly, a reference data bank that comprises reference expression levels of a target gene combination originated from at least one in vitro normal bladder specimen is stored in a system. Next, expression levels of the target gene combination of a biological specimen originated from in vitro aggressive bladder cancer of the individual are detected. And then, one of the expression levels of the biological specimen is compared with a corresponding reference expression level of the target gene combination of a reference database respectively, for obtaining a difference and a risk score. Subsequently, a risk score sum of at least two of the target gene combination of the biological specimen is calculated. A program executes the aforementioned steps of comparing one of the expression levels with a corresponding reference expression level of the target gene combination of a reference database respectively, obtaining a difference and a risk score, and calculating a risk score sum of at least two of the target gene combination of the biological specimen, for implementing the method on a computer system. In some examples, the program used for executing the computer-implemented method for in vitro predicting survival time of an individual with bladder cancer after surgery from an individual's biological sample can be also called as a biomarker screening software.

In other embodiments, the computer program can be applied to a system for in vitro predicting survival time of an individual with bladder cancer after surgery from an individual's biological sample. The system includes a detecting module, a processing module, an identifying module and a controlling module. In this embodiment, the detecting module can include a detecting device, a reaction solution, nucleic acid probes and/or antibodies. The detecting module can be performed in combination with conventional detection reagents, a detecting equipment, a microarray chip and so on, which are common knowledge in the technical field of the present invention instead of reciting in detail. The nucleic acid probes and/or the antibodies react with the target gene combination of the biological sample for generating expression levels that are detected by the detecting device. The aforementioned biological specimen can be originated from in vitro aggressive bladder cancer of an individual, and the target gene combination is selected from at least two of the group consisting of PPT2, ARMH4, P4HB, SLC1A6 and ARID3A, a fragment, a homologue, a variant and a derivative thereof. The processing module can be coupled to the detecting module, for receiving the expression levels from the detecting module and comparing one of the expression levels with a corresponding reference expression level of the target gene combination of a reference database respectively, thereby obtaining a difference and a risk score. The processing module also can calculate a risk score sum of at least two of the target gene combination of the biological specimen, and the risk score of the one is given as 1 when an absolute value of the difference is equal to or more than a first threshold that is more than at least 5% of the reference expression level. The identifying module can be coupled to the processing module, for identifying the individual from the risk score sum. When the risk score sum is equal to or greater than a second threshold that is 1 or 2, the individual having the biological specimen is classified to a high risk group, the high risk group is defined by less 25 months of an averaged survival time after surgery of the individual with 1 or 2 of the second threshold, or by less 10 months of an averaged survival time after surgery of the individual with 3 of the second threshold. The controlling module can be coupled to the detecting module, the processing module and the identifying module. The system executes a program that includes instructions for activating the detecting module, the processing module and the identifying module. In the above embodiment, the system optionally includes a pretreating module that is coupled to the detecting module, for providing a nucleic acid sample and/or a protein sample of the biological specimen.

Thereinafter, it will be understood that particular cancer subjects, biological sample, target gene combinations, detection methods, determination criteria, aspects, examples and embodiments described hereinafter are shown by way of illustration and not as limitations of the invention. The principal features of this invention can be employed in various embodiments without departing from the scope of the invention. Thus, one skilled in the art can easily ascertain the essential characteristics of the present invention and, without departing from the spirit and scope thereof, can make various changes and modifications of the invention to adapt it to various usages and conditions.

### Example 1

1.1 Use and Statistical Analysis of Data from The Cancer Genome Atlas (TCGA)

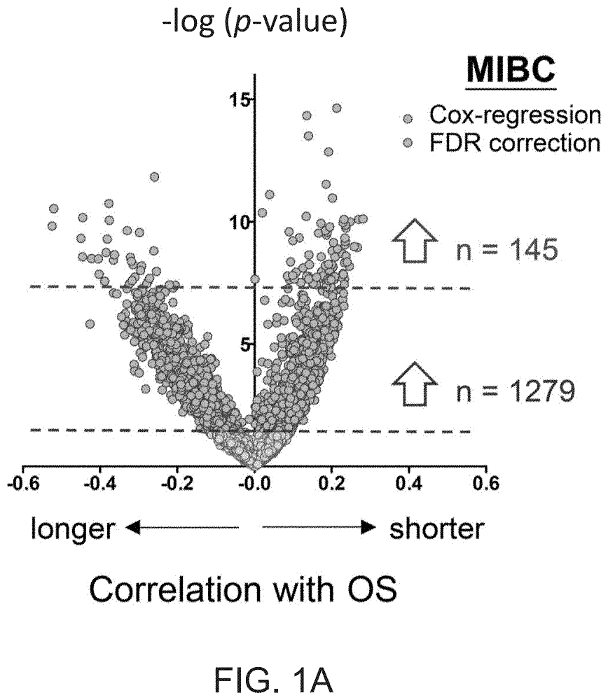

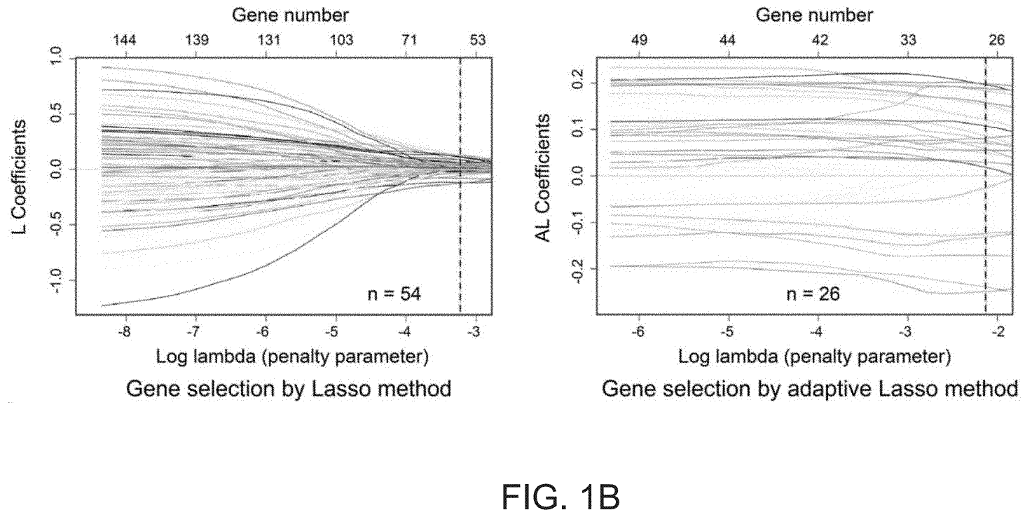

Reference was made to FIGS. 1A and 1B. FIG. 1A showed the computer-implemented method for in vitro predicting survival time of an individual with bladder cancer after surgery from an individual's biological sample according to an embodiment of the present invention, which involved a univariate analysis result of the bladder cancer patient database using Cox regression analysis and FDR correction. FIG. 1B showed the computer-implemented method for in vitro predicting survival time of an individual with bladder cancer after surgery from an individual's biological sample according to an embodiment of the present invention, which involved a multivariate and high-dimensional analysis result of genes in FIG. 1A using the Lasso algorithm (the left panel of FIG. 1B) and the adaptive Lasso algorithm (the right panel of FIG. 1B).

Firstly, a bladder cancer dataset (TCGA, Cell 2017) having the most samples (n=413) in the bladder/urinary tract in the cBioPortal online database was taken in this Example, the patients with bladder cancer that had complete RNA expression and clinical data were adopted (n=408) among these, and the patients with bladder cancer that had events of overall survivals (n=405) were further chosen (n=367). And then, the gene lists (n=18883) excluded NA (not applicable/not available) value from 20,435 gene lists of RNA expressions of patients (n=18883). Next, the survival-related genes (n=1279) were calculated by univariate Cox regression analysis (p-value<0.05) from the first screening result (n=7204, the screening criteria: the expression rate>5%, Z-score>2), and then corrected by False Discovery Rate (FDR) (adjusted p value<0.05), so as to remove erroneous genes as much as possible (as shown in FIG. 1A). Subsequently, the genes (n=145) corrected by FDR were uploaded to the high-dimensional analysis of molecular alterations in cancer (HD-MAC) of the online shiny statistical analysis platform, calculated by the Cox proportional hazards model (CoxPH Model) of the platform, categorized and screened by various normalization model results (Ridge, Lasso or Adaptive Lasso).

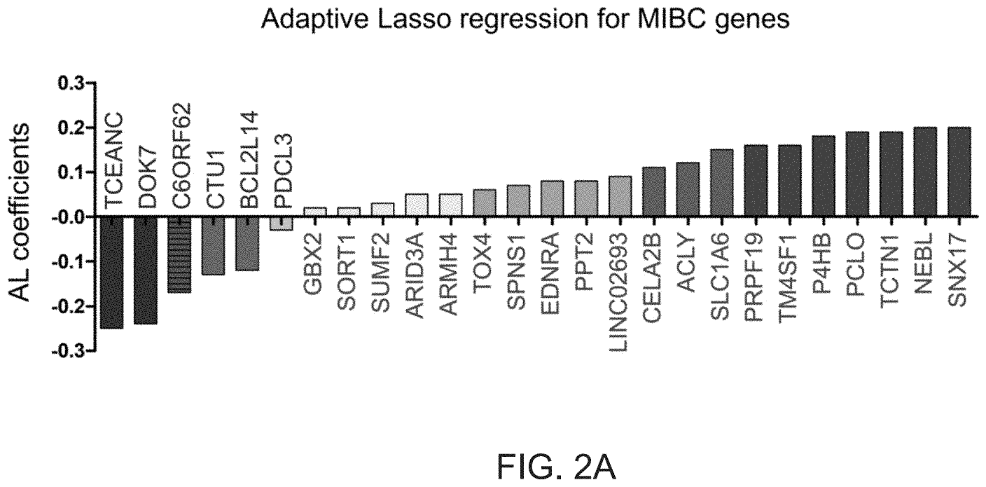

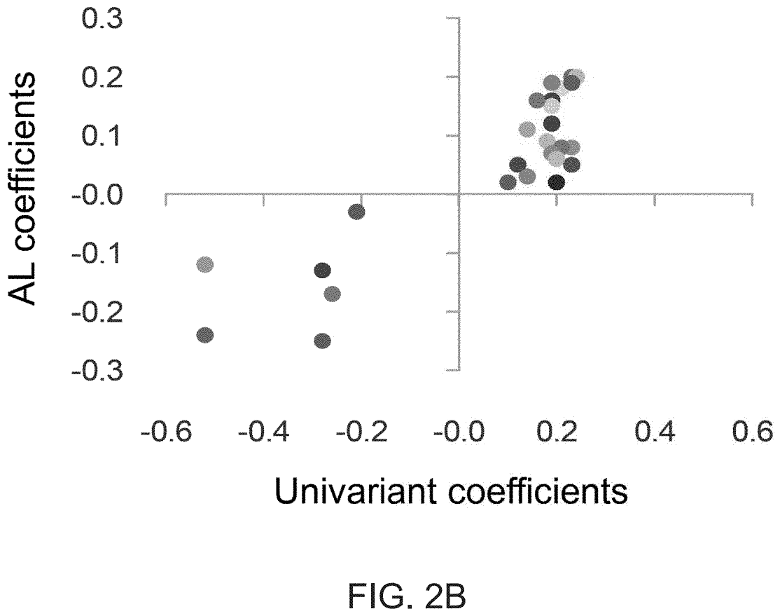

The 145 genes in FIG. 1A were analyzed by the multivariate high-dimensional analysis using the Lasso algorithm and the adaptive Lasso algorithm, so as to screen out 54 and 26 gene respectively, as shown in the left and right panels of FIG. 1B. The aggressive cancer risk scores of the 26 genes screened from FIG. 1B were analyzed and revealed in FIG. 2A and FIG. 2B. Reference was made to FIG. 2B, which showed a scatter plot representing univariate and multivariate predicted risk coefficients of the 26 genes screened from FIG. 2A. As the result of FIG. 2B, the computer-implemented method for predicting survival time of an individual with bladder cancer after surgery from an individual's biological sample, the risk scores of the predicted aggressive cancer and the monovariate analysis were dispersed in the Quadrant 1 (i.e., twenty genes with positive AL coefficients as shown in FIG. 2A) and Quadrant 3 (i.e., six genes with negative AL coefficients as shown in FIG. 2B). FIG. 2B showed the consistent results of positive correlation and consistent tendency with the 26 genes screened from FIG. 2A, either in the univariate predicted risk coefficients or multivariate predicted risk coefficients.

### 1.2. Predictability of the Model

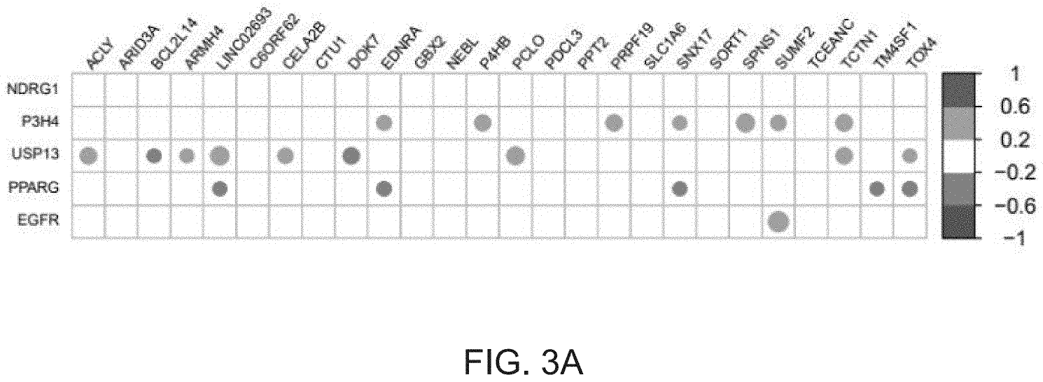

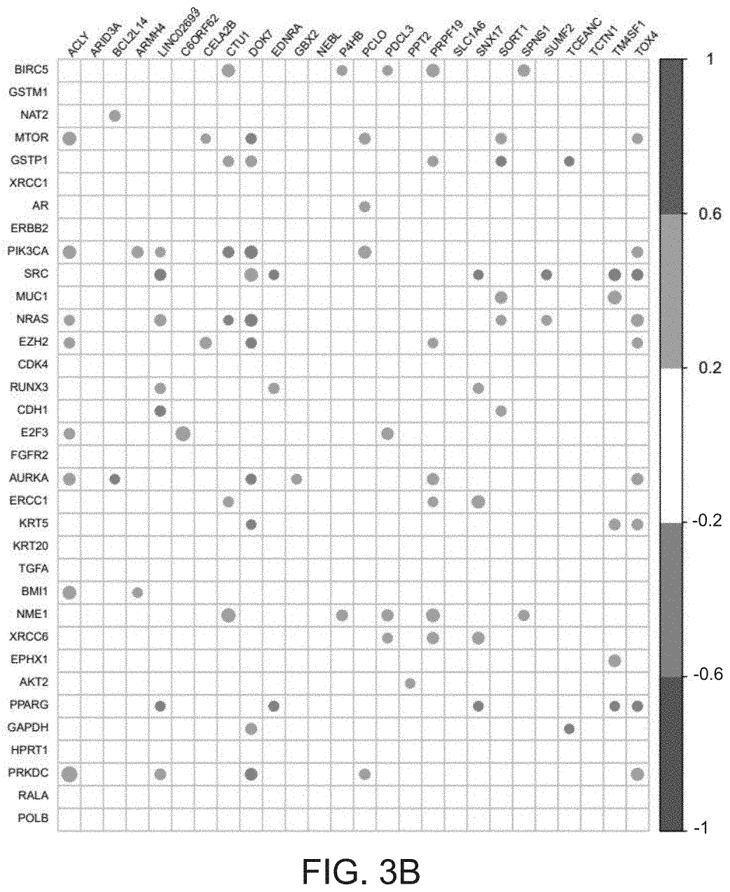

For the purpose of realizing the predictability of the model, this Example proposed to find genes that were related to bladder cancer, overexpressed and reported in the journals. According to the first screening criteria (i.e., expression rate>5%, Z-score>2), two gene lists (n=5, also called as 5g, known oncogenes with abnormal overexpression, as shown in the vertical axis of FIG. 3A; n=34, also called as 34g, known oncogenes with activating mutation, as shown in the vertical axis of FIG. 3B) were overexpressed in the bladder cancer, respectively. The two gene lists and the 26 genes screened from FIG. 2A [HDMAC 26g (MIBC 367p), as shown in the horizontal axis of FIG. 3B] were subjected to a correlation analysis, the results of which were shown in FIG. 3A and FIG. 3B, respectively.

Reference was made to FIGS. 3A and 3B. FIG. 3A showed the 26 genes screened from FIG. 2A according to an embodiment of the present invention. FIG. 3B showed a matrix diagram of correlation analysis of known activating mutations in the oncogene list according to an embodiment of the present invention. The results of FIGS. 3A and 3B indicated that the 26 genes screened from FIG. 2A were associated with genes published in several journals, and the one of the 26 genes could be simultaneously associated with many genes.

In addition, 26 genes screened from by the computer program for in vitro predicting survival time of an individual with bladder cancer after surgery from an individual's biological sample of FIG. 2A had higher gene correlation than the gene randomly screened from the 18883 genes, as shown in Table 1.

TABLE 1

| | | Averaged numbers greater than the given | | |
|---|---|---|---|---|
| | | correlation coefficient (ρ) | | |
| Positive and | ρ | 18883 g | 5 g | 34 g |
| negative | 0.3 | 0.482 | 1 | 0.765 |
| correlation | 0.35 | 0.241 | 0.6 | 0.324 |
| | 0.4 | 0.126 | 0.2 | 0.088 |
| | 0.45 | 0.069 | 0 | 0.059 |

### 1.3 Validation of Gene Expression Omnibus (GEO)

The genes (n=26, as shown in FIG. 2A) screened from Section 1.1 were further validated whether a consistent result could be found between gene overexpression and the survival rate of the patients with bladder cancer in the gene expression omnibus (GEO).

In this Example, a survival rate of a MIBC patient dataset (GSE13507) from GEO database and the genes (n=26) screened from FIG. 2A were analyzed according to the condition of overall survivals/MIBC (n=62). As a result, only 24 genes could be found in the GEO gene list, whereas C6ORF62 and TCEANC could not be found in the GEO gene list.

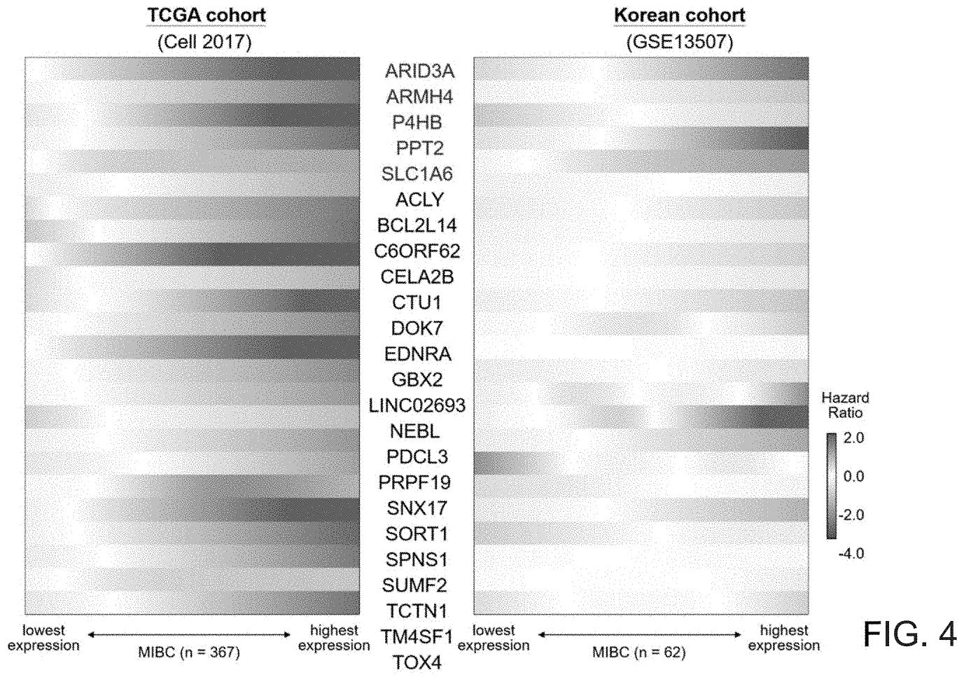

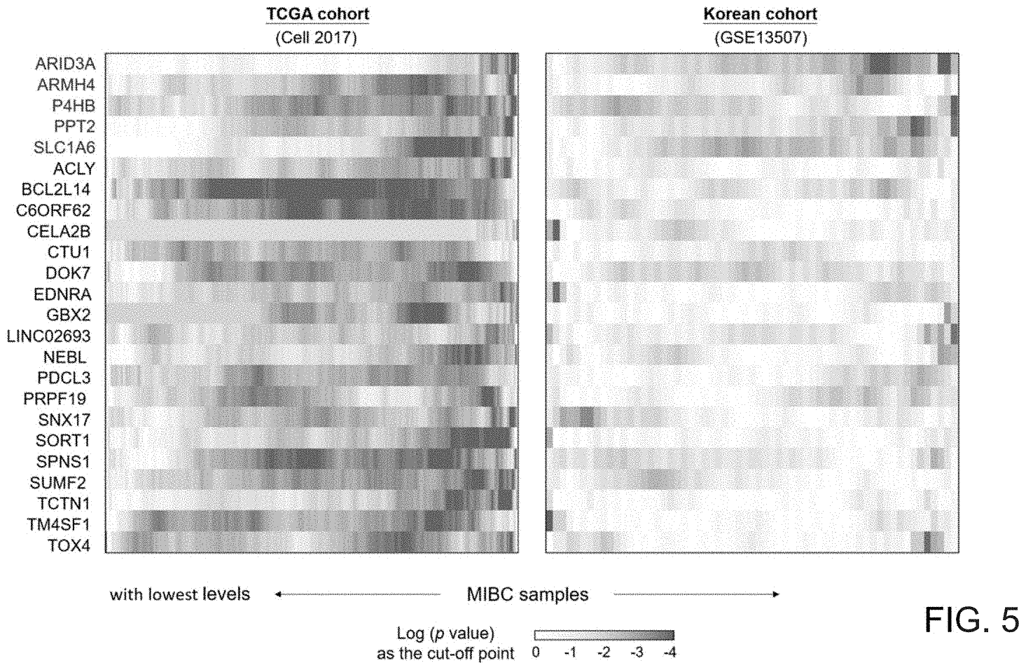

The data of the MIBC patient (GSE13507) of GEO database and the patient (cell 2017) with bladder cancer of TCGA database were analyzed, for assessing the overall risk trend and producing the heat map (as shown in FIG. 4) of TCGA and GEO risk scores. Also, the cutoff points (as shown in FIG. 5) were chosen according to gene expression levels of patient ranking from small to large and Cox proportional-hazards model (CoxPH Model).

Reference was made to FIG. 4, which showed a heat map representing genetic risk coefficients of MIBC samples of GEO database (GSE13507) (the right panel) and TCGA database (the left panel) according to an embodiment of the present invention, in which Red-to-blue color scheme represented the expression level of a single gene, and blue to red color from the left side to the right side of the single gene represented a positive correlation between the gene expression and the genetic risk. As shown in the result of FIG. 4, after gene risk scores of MIBC sample of GEO database (GSE13507) (the right panel) were compared with the heat map of TCGA database (the left panel), so that the genes having the consistent and significantly increasing (i.e., from blue to red) risk scores were chosen, which included the five genes of PPT2, ARMH4, P4HB, SLC1A6 and ARID3A, and their gene expression levels were correlated positively (i.e., from blue to red).

Reference was made to FIG. 5, which showed a ranking heat map of the significance [log (p value)] of gene expression levels of MIBC samples of GEO database (GSE13507) (the right panel) and TCGA database (the left panel) according to an embodiment of the present invention, both of which were analyzed by Cox proportional-hazards model (CoxPH Model), and a cutoff point was defined as the point with the most significant split of an gene expression level of a single gene.

As shown in the result of FIG. 5, the expression levels of the genes of patients were ranked from low to high, the cutoff point of each gene was found according to CoxPH Model, and five genes of PPT2, ARMH4, P4HB, SLC1A6 and ARID3A had similar cutoff points in the GEO database (the right panel) and TCGA database (the left panel) (e.g., p-value<0.05 of the blue squares in the percentile rank).

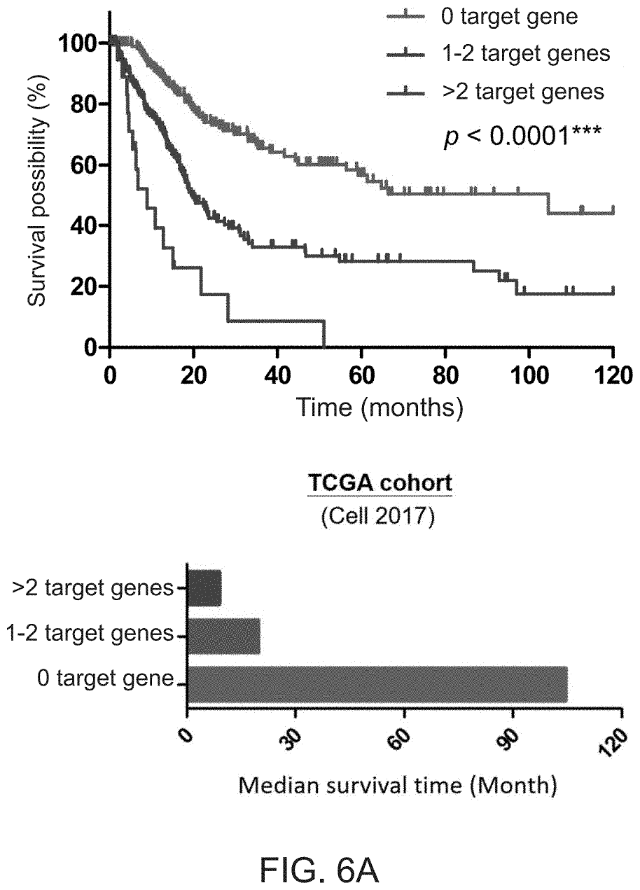

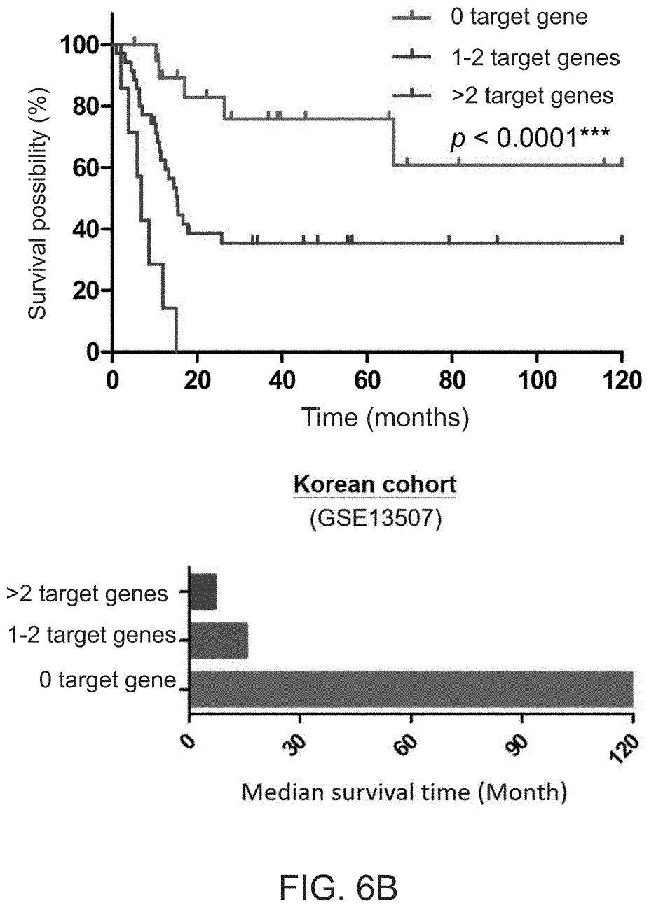

In addition, PPT2, ARMH4, P4HB, SLC1A6 and ARID3A were subjected to Kaplan-Meier survival analysis and the result was shown in FIGS. 6A to 6B.

Reference was made to FIGS. 6A to 6B, which respectively showed Kaplan-Meier survival curves and survival rates of genes screened from TCGA database (FIG. 6A) and GEO database (FIG. 6B) according to an embodiment of the present invention. As shown in the results of FIGS. 6A to 6B, among the five genes of the target gene combination, when each additional gene with excess expression was detected in a patient with bladder cancer, such patient had a poorer clinical prognosis (as shown in FIG. 6B). The survival rate of the genes screened by TCGA was analyzed in this example, and the same or similar results were obtained (as shown in FIG. 6A).

As shown in previous research, SLC1A5 in the SLC family was associated with glutamate and cancer, but there was no correlation between them found in the analysis of SLC1A5 and the survival rate of patients with bladder cancer. However, this Example proved that genes of ARID3A, ARMH4, P4HB, PPT2, SLC1A6 and so on were correlated with the survival rate of patients with bladder cancer. Moreover, the aforementioned results confirmed that when at least two of the target gene combination of PPT2, ARMH4, P4HB, SLC1A6 and ARID3A were detected in the biological sample, the survival rate of the clinical prognosis of the individual corresponding to the biological sample was actually poor, and an averaged survival time after surgery of the individual would be less than 25 months.

### 1.4. Coimmunoprecipitation and Western Blot Analysis

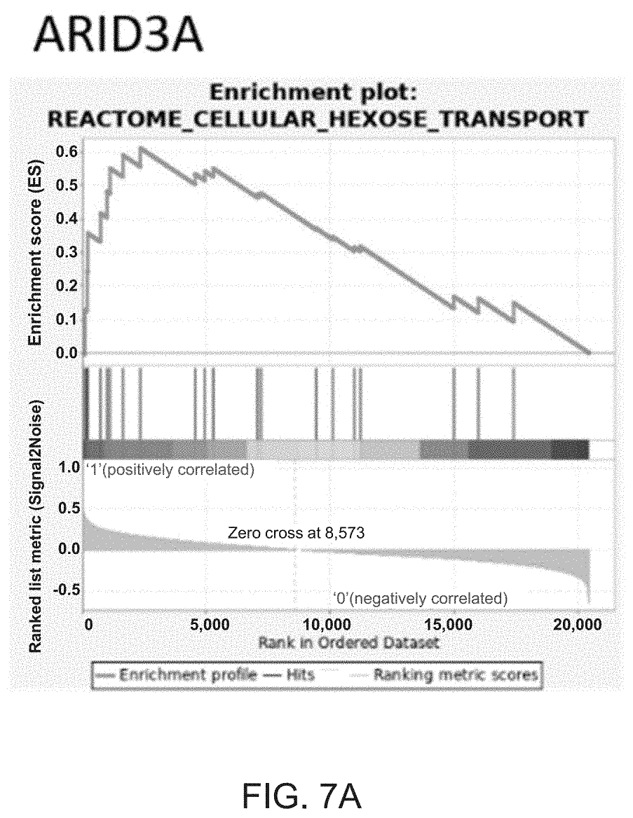

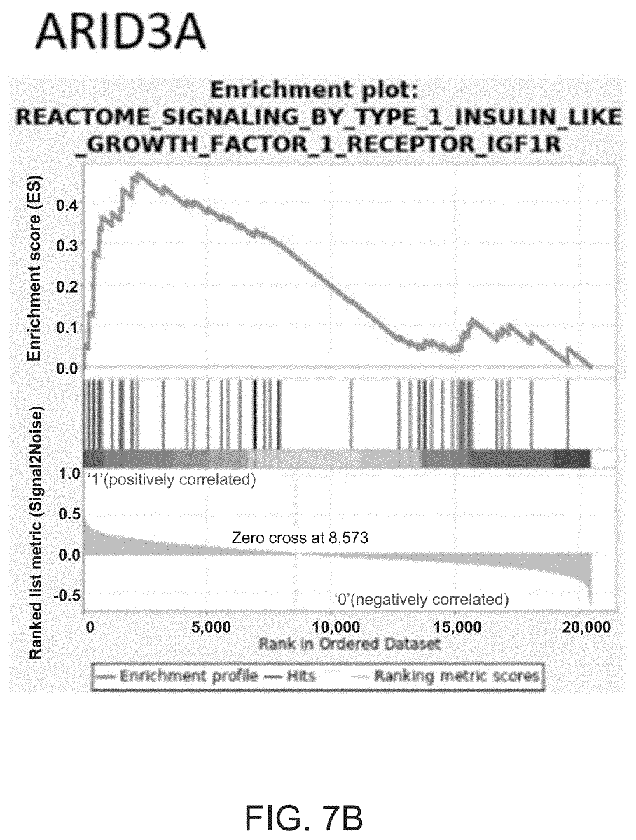

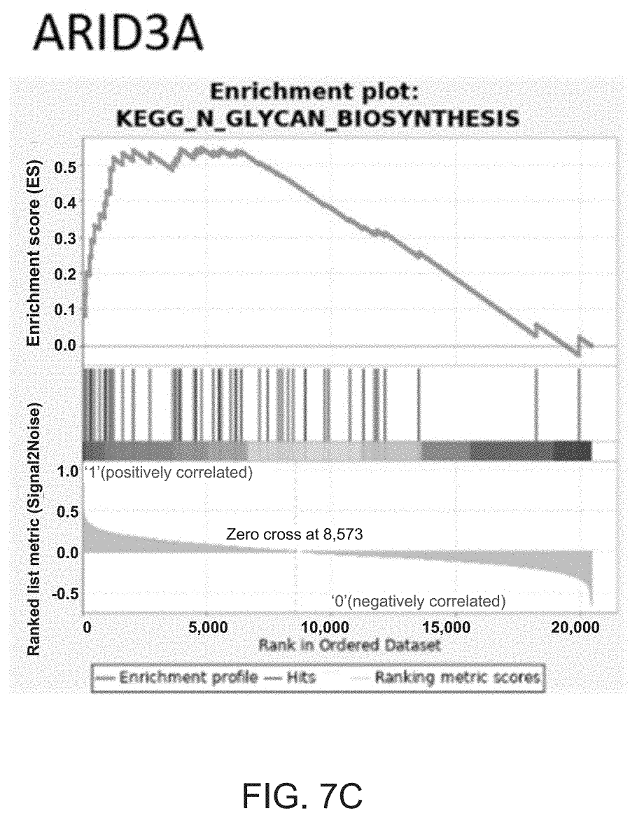

Reference was made to FIGS. 7A to 7C, which respectively showed several gene enrichment plots related to ARID3A overexpression according to the computer-implemented method for in vitro predicting survival time of an individual with bladder cancer after surgery from an individual's biological sample of the present invention. As shown in the GSEA analysis results of FIGS. 7A to 7C, ARID3A would participate in the cellular hexose transport (FIG. 7A), signaling by type-1 insulin-like growth factor-1 receptor (IGF1R) (FIG. 7B), and N-glycan biosynthesis (FIG. 7C). However, this Example proved that ARID3A gene was correlated with energy/nutritional homeostasis, cancer-related signaling, and glycome alterations for TME remodeling of the individual with bladder cancer.

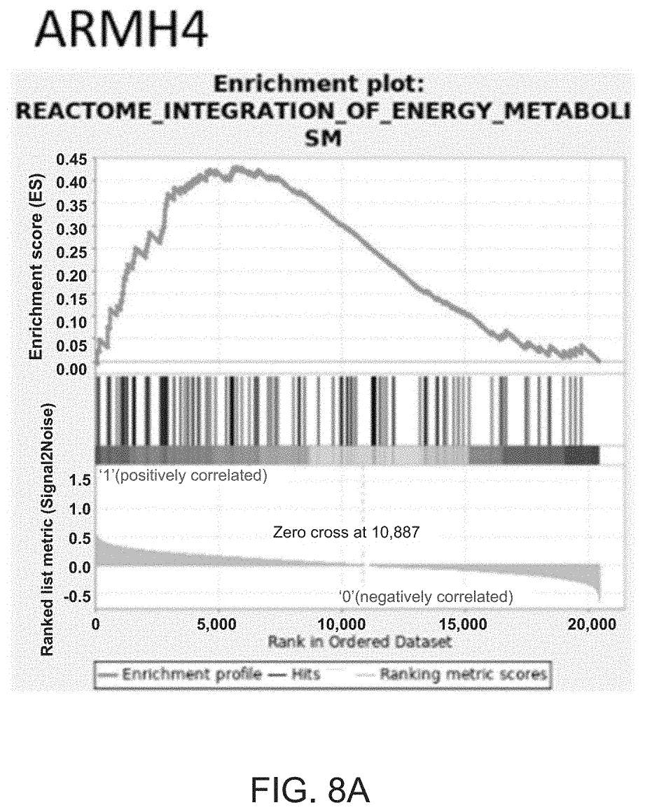

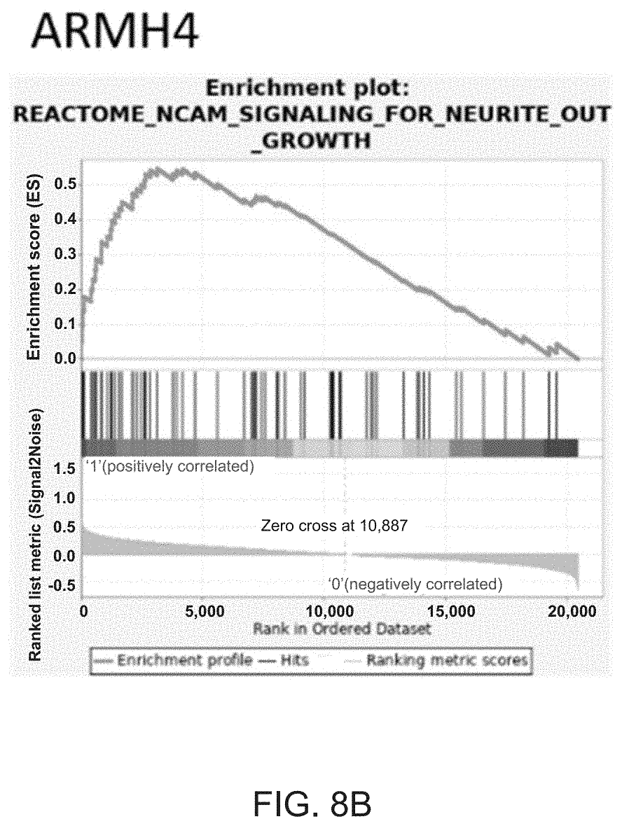

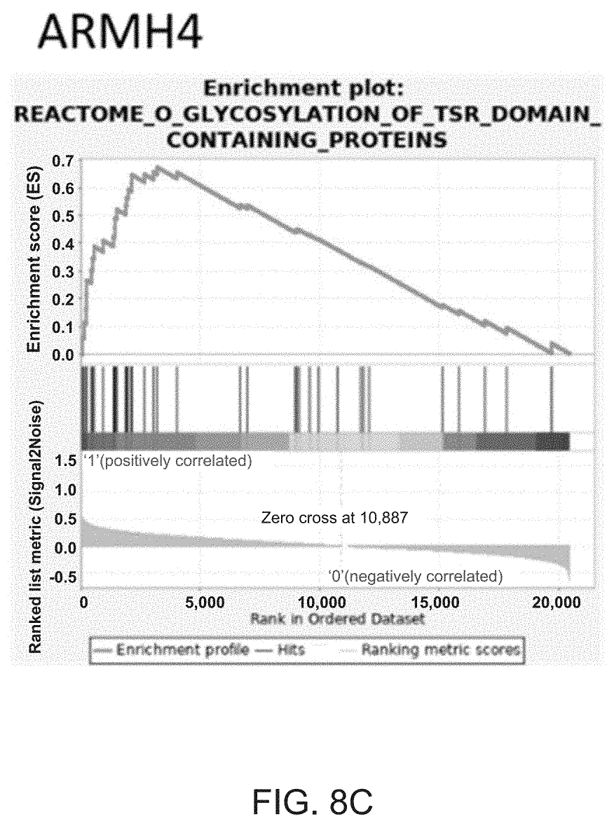

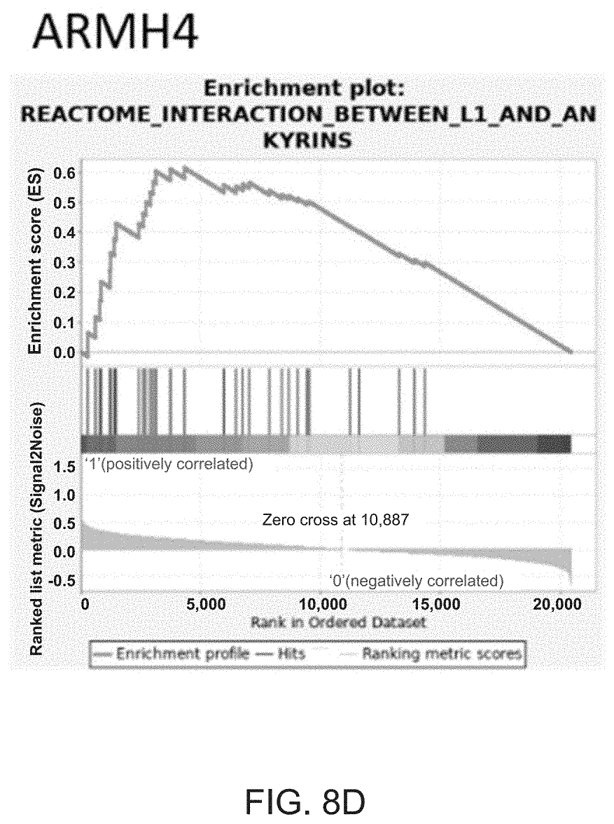

Reference was made to FIGS. 8A to 8D, which respectively showed several gene enrichment plots related to ARMH4 overexpression according to the computer-implemented method for in vitro predicting survival time of an individual with bladder cancer after surgery from an individual's biological sample of the present invention. As shown in the GSEA analysis results of FIGS. 8A to 8D, ARMH4 involved in the integration of energy metabolism (FIG. 8A), NCAM (neural cell adhesion molecule) signaling for neurite outgrowth (FIG. 8B), O-glycosylation of TSR (thrombospondin type 1 repeat) domain containing proteins (FIG. 8C), and the interaction between L1 and ankyrins (FIG. 8D). This Example proved that ARMH4 gene was correlated with energy, neuronal signaling, glycome alterations for TME remodeling, and cancer-related signaling of the individual with bladder cancer.

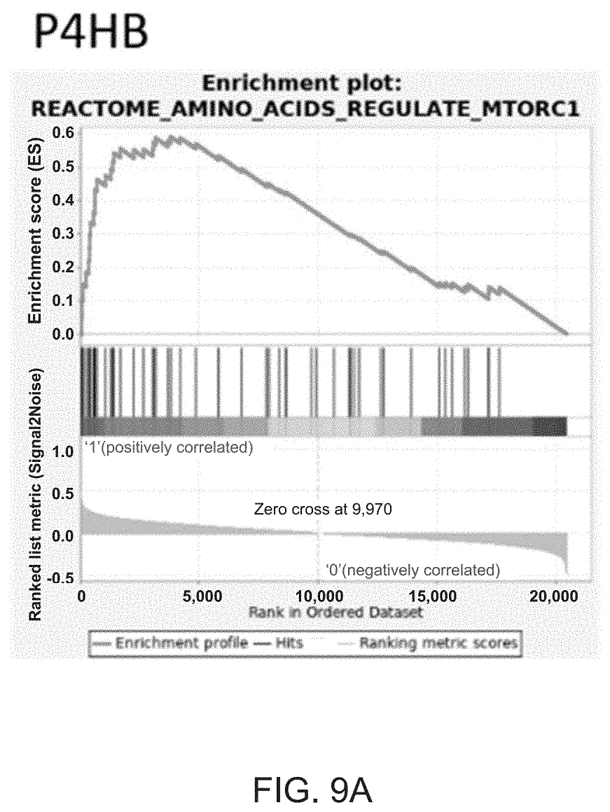

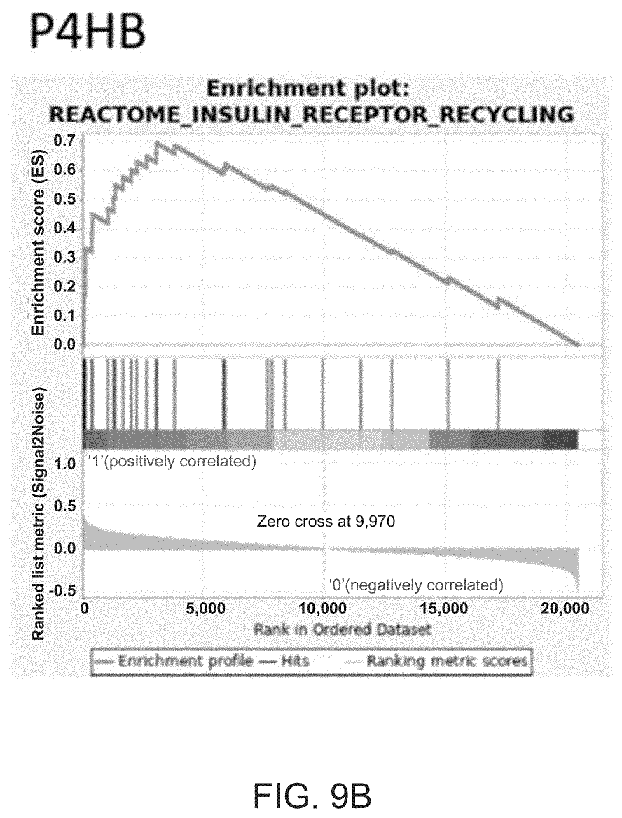

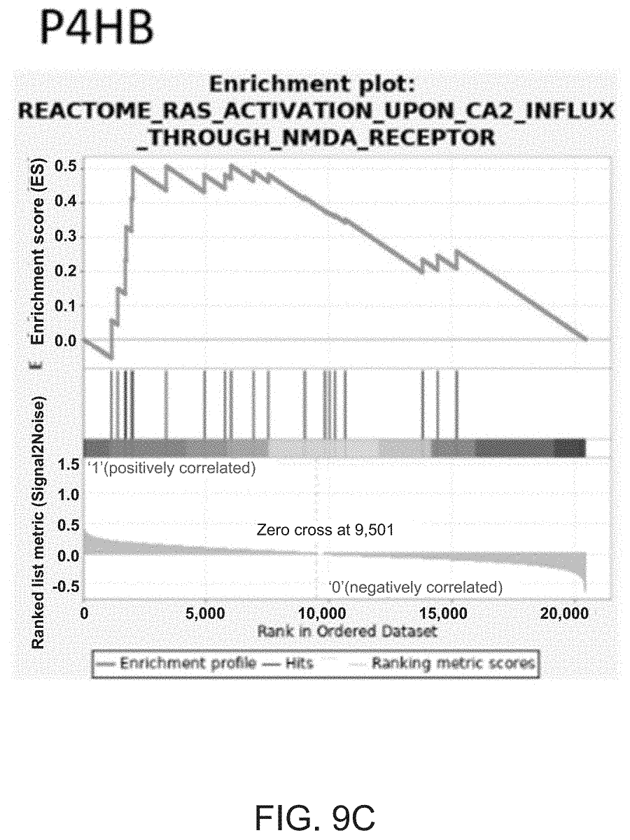

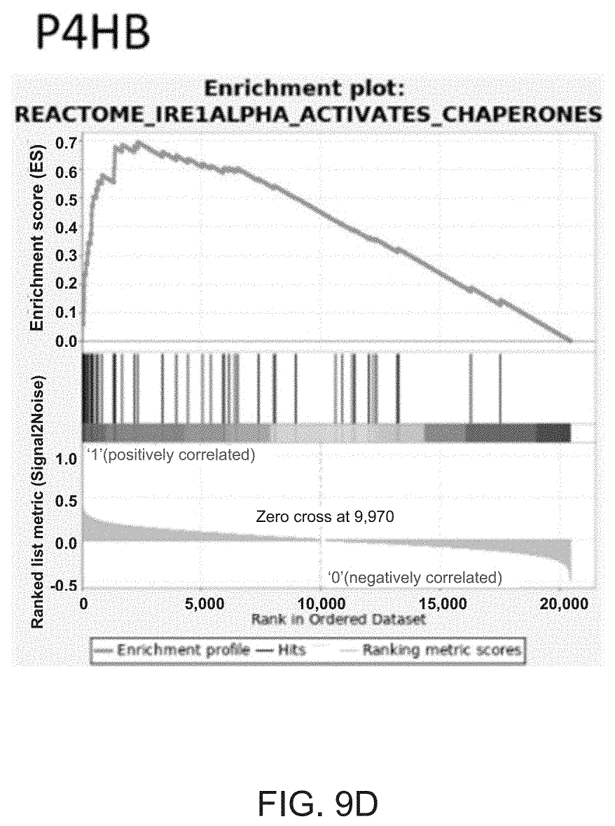

Reference was made to FIGS. 9A to 9D, which respectively showed several gene enrichment plots related to P4HB overexpression according to the computer-implemented method for in vitro predicting survival time of an individual with bladder cancer after surgery from an individual's biological sample of the present invention. As shown in the GSEA analysis results of FIGS. 9A to 9D, P4HB was involved in the amino acid regulating mTORC1 (FIG. 9A); insulin receptor recycling (FIG. 9B); RAS activation upon CA2 in flux through NMDA receptor (FIG. 9C); and IRE1alpha activating chaperones (FIG. 9D). This Example proved that P4HB gene was correlated with energy/nutritional homeostasis, glycome alterations for TME (tumor microenvironment) remodeling, and regulation in cell death of the individual with bladder cancer.

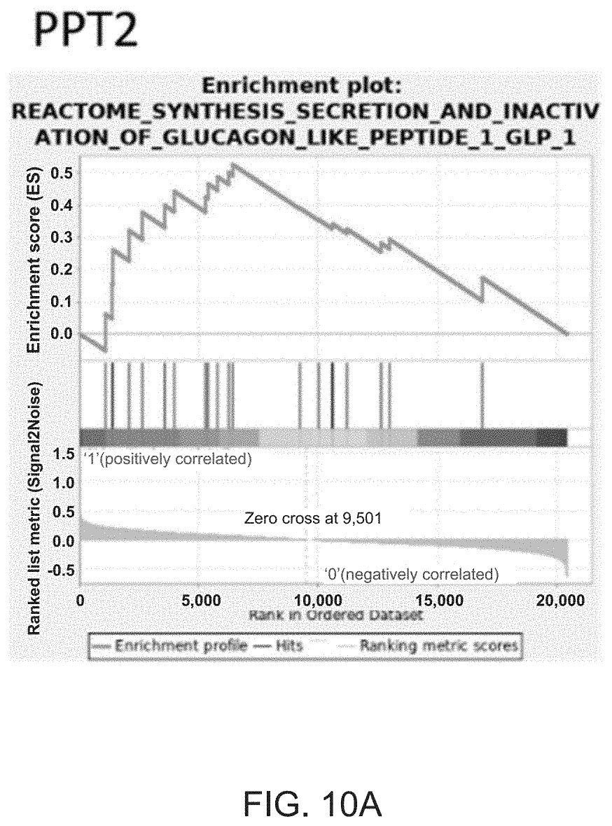

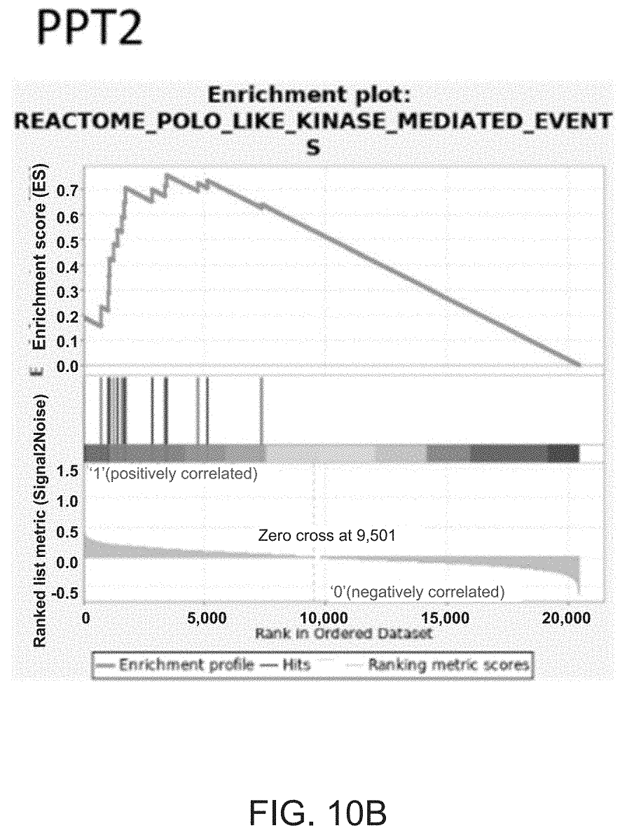

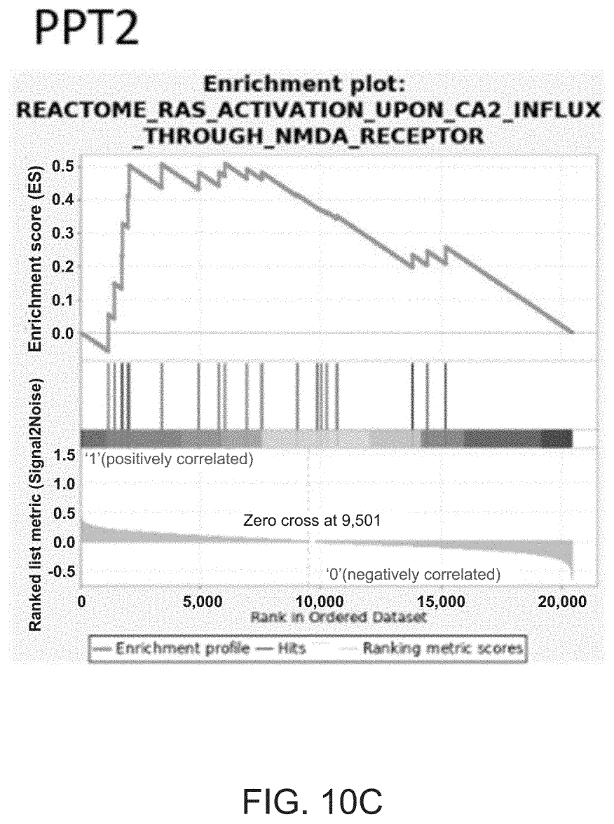

Reference was made to FIGS. 10A to 10C, which respectively showed several gene enrichment plots related to PPT2 overexpression according to the computer-implemented method for in vitro predicting survival time of an individual with bladder cancer after surgery from an individual's biological sample of the present invention. As shown in the GSEA analysis results of FIGS. 10A to 10C, PPT2 was involved in the synthesis, secretion and inactivation of glucagon-like peptide 1 (GLP 1) (FIG. 10A); POLO-like kinase mediated events (FIG. 10B); and RAS activation upon CA2 in flux through NMDA receptor (FIG. 10C). This Example proved that PPT2 gene was correlated with energy/nutritional homeostasis, cancer-related signaling, and glycome alterations for TME remodeling of the individual with bladder cancer.

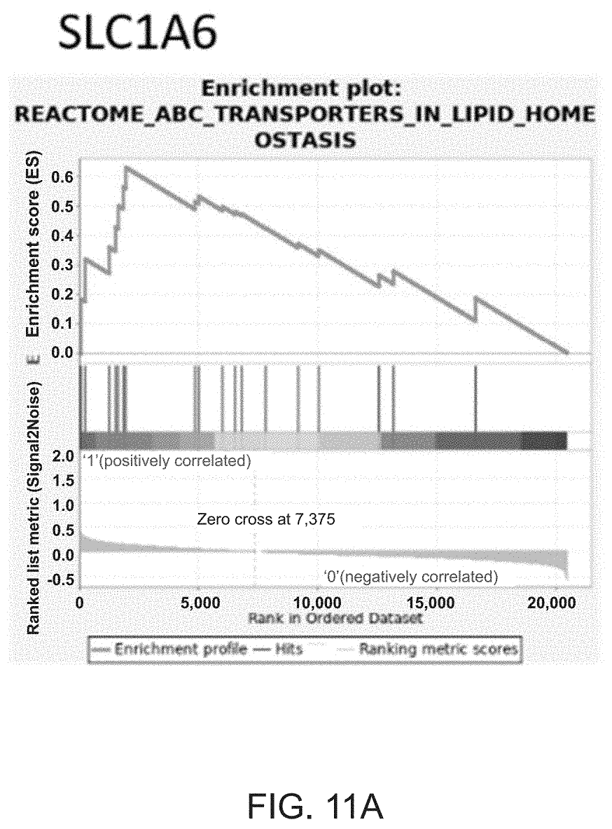

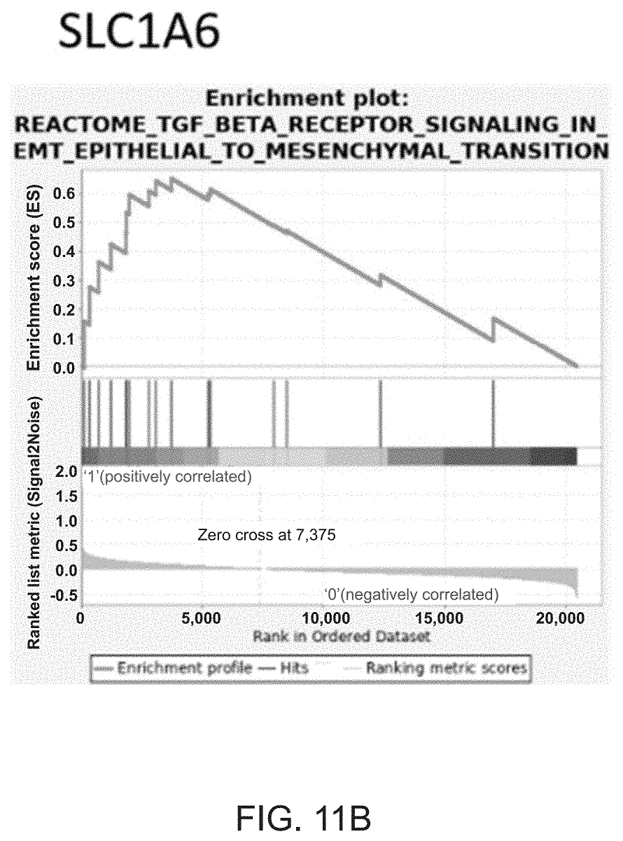

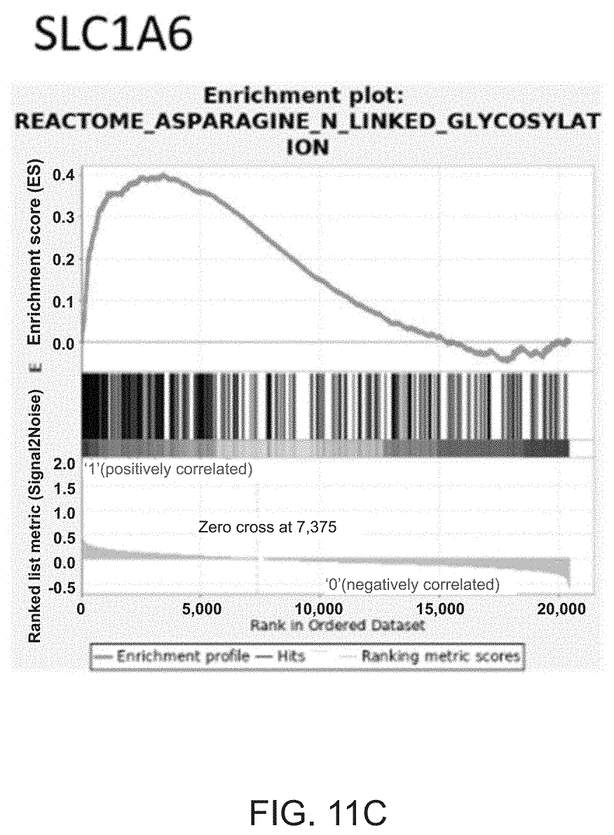

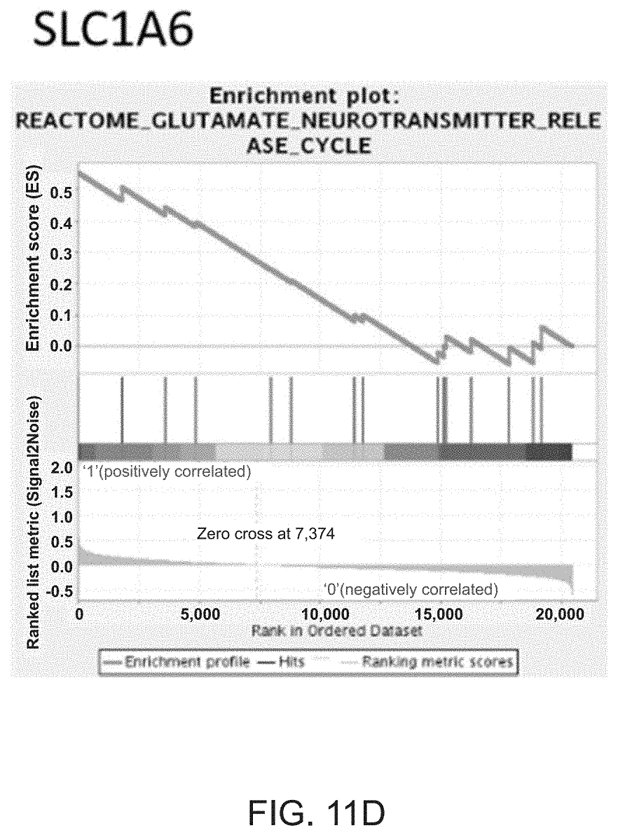

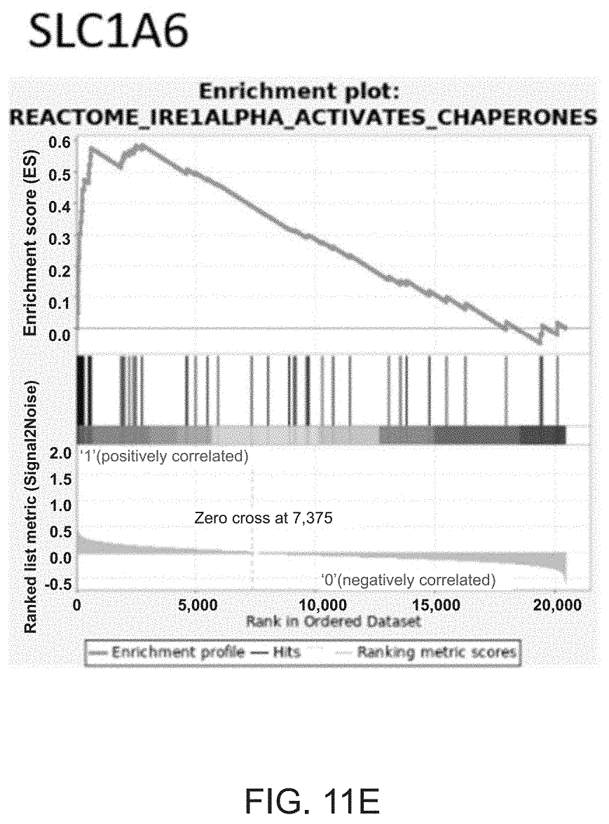

Reference was made to FIGS. 11A to 11E, which respectively showed several gene enrichment plots related to SLC1A6 overexpression according to the computer-implemented method for in vitro predicting survival time of an individual with bladder cancer after surgery from an individual's biological sample of the present invention. As shown in the GSEA analysis results of FIGS. 11A to 11E, SLC1A6 was involved in ABC transporters in lipid homeostasis (FIG. 11A); TGF-beta receptor signaling in EMT (epithelial to mesenchymal transition) (FIG. 11B); asparagine N-linked glycosylation (FIG. 11C); glutamate neurotransmitter release cycle (FIG. 11D); and RAS activation upon CA2 in flux through NMDA receptor (FIG. 10C); glutamate neurotransmitter release cycle (FIG. 10D); and IRE1alpha activating chaperones (FIG. 11E). Previous researches showed that SLC1A5 in the SLC family was related to glutamate and cancer, but SLC1A5 and the survival rate of bladder cancer patients were analyzed and found no correlation between them. This Example proved that SLC1A6 gene was correlated with energy/nutritional homeostasis, glycome alterations for TME remodeling, glutamate signaling and neuronal signaling pathways, and regulation in cell death.

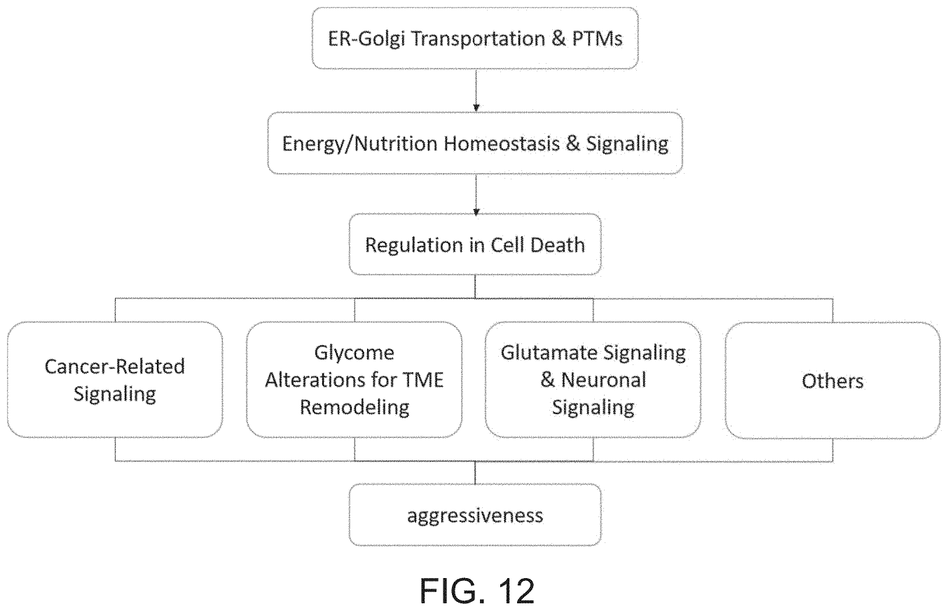

Reference was made to FIG. 12, which showed various metabolic pathways associated with aggressive bladder cancer by combining 5 target genes according to several embodiments of the present invention. As shown in the result of FIG. 12, the target gene combination was correlated with transport through ER (endoplasmic reticulum)—Golgi transportation and PTMs (post-translational modifications), leading in the influence on energy/nutritional homeostasis and signaling, as well as regulation in cell death. Those result further affect cancer-related signaling, glycome alterations for TME remodeling, glutamate signaling, neuronal signaling and others, thereby affecting the cancer invasion.

In FIG. 12, the others as aforementioned could be exemplified as follows. ARMH4 was also correlated with myogenesis, elastic fiber formation, molecules associated with elastic fibers, and vasopressin regulated water reabsorption. P4HB was also correlated with Vibrio cholerae infection. PPT2 was also correlated with processing of capped intronless pre-mRNA, and epigenetic regulation of gene expression. ARID3A was also correlated with NO (nitric oxide). SLC1A6 was also correlated with defects of contact activation system (CAS) and kallikrein kinin system (KKS).

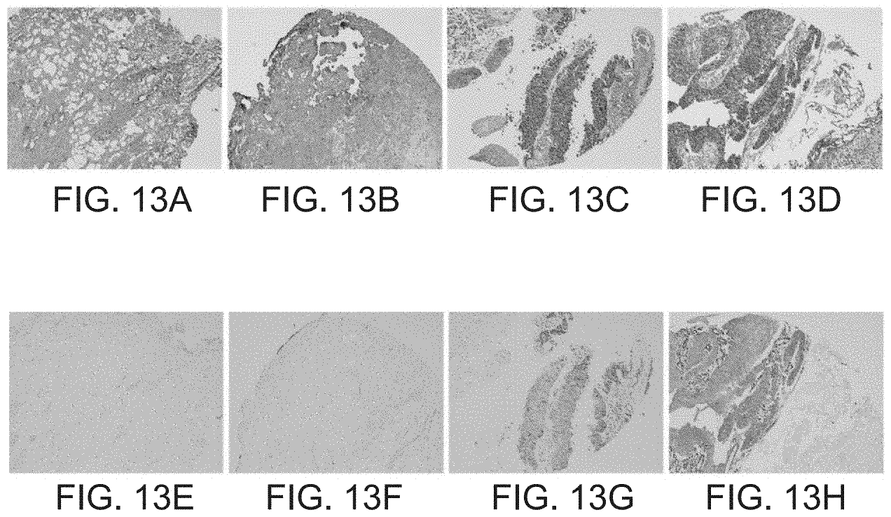

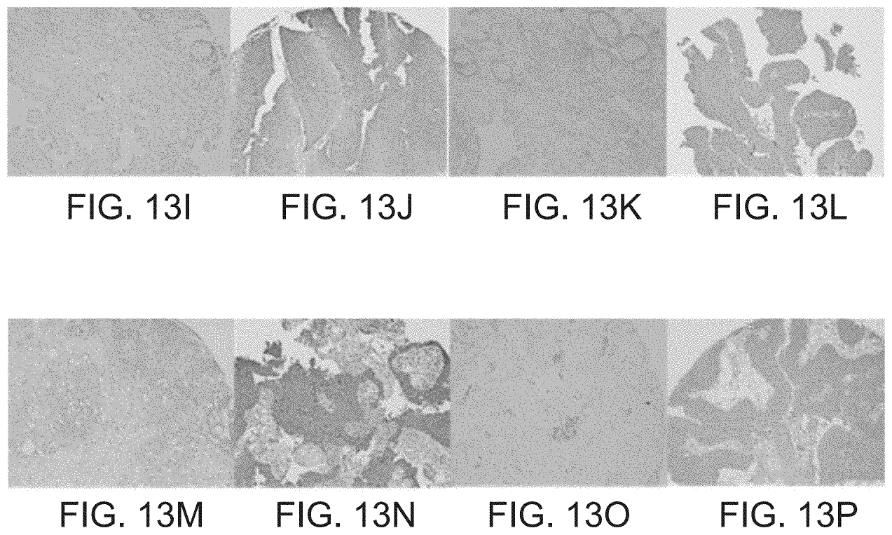

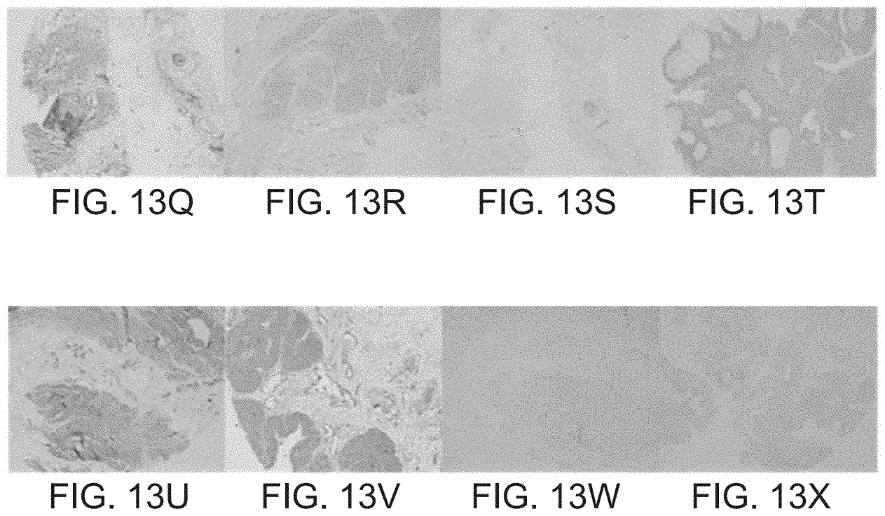

In addition, reference was made to FIGS. 13A to 13D, which showed histochemical staining images of normal bladder tissue sections (FIGS. 13A to 13B) and aggressive bladder cancer tissue sections (FIGS. 13C to 13D) with 4-fold magnification according to several embodiments of the present invention, all of which were obtained by hematoxylin and eosin (H&E) staining.

Reference was made to FIGS. 13E to 13X, which showed histochemical staining images of normal bladder tissue sections (FIG. 13E, FIG. 13F, FIG. 13I, FIG. 13K, FIG. 13M, FIG. 13O, FIG. 13Q, FIG. 13S, FIG. 13U, FIG. 13W) and aggressive bladder cancer tissue sections (FIG. 13G, FIG. 13H, FIG. 13J, FIG. 13L, FIG. 13N, FIG. 13P, FIG. 13R, FIG. 13T, FIG. 13V, FIG. 13X) with 4-fold magnification according to several embodiments of the present invention. FIGS. 13E to 13H were IHC staining images detected by anti-SLC1A6 antibody (Brand name: Elabscience, 1:100 of dilution ratio); FIG. 13I, FIG. 13J, FIG. 13M and FIG. 13N were IHC staining images detected by anti-P4HB antibody (Brand name: Elabscience, 1:100 of dilution ratio); FIG. 13L, FIG. 13L, FIG. 13O and FIG. 13P were IHC staining images detected by anti-ARID3A antibody (Brand name: Elabscience, 1:100 of dilution ratio); FIG. 13Q, FIG. 13R, FIG. 13U and FIG. 13V were IHC staining images detected by anti-PPT2 antibody (Brand name: Elabscience, 1:100 of dilution ratio); and FIG. 13S, FIG. 13T, FIG. 13W and FIG. 13X were IHC staining images detected by anti-ARMH4 antibody (Brand name: Elabscience, 1:100 of dilution ratio).

According to the immunohistochemical staining results of FIGS. 13E to 13X, the tissues existed significant differences between the individuals with bladder cancer (FIG. 13C, FIG. 13D, FIG. 13G, FIG. 13H, FIG. 13J, FIG. 13L, FIG. 13N, FIG. 13P, FIG. 13R, FIG. 13T, FIG. 13V, FIG. 13X) and the normal individuals (FIG. 13A, FIG. 13B, FIG. 13E, FIG. 13F, FIG. 13I, FIG. 13K, FIG. 13M, FIG. 13O, FIG. 13Q, FIG. 13S, FIG. 13U, FIG. 13W).

In summary, specific cancers, specific biological samples, specific target gene combinations, specific analysis models or specific evaluating methods are exemplified for clarifying the method and the kit, the computer-implemented method and the system for in vitro predicting survival time of an individual with bladder cancer after surgery from an individual's biological sample. However, as is understood by a person skilled in the art, other cancers, other biological samples, other target gene combinations, other analysis models or other evaluating methods can be also adopted in the method and the kit, the computer-implemented method and the system for in vitro predicting survival time of an individual with bladder cancer after surgery from the individual's biological sample without departing the spirit and scope of the present invention rather than being limited as aforementioned. For example, the aforementioned target gene combinations can be added with other genes as molecular typing markers, for optimizing the method and the kit, the computer-implemented method and the system, thereby elevating the accuracy for predicting an averaged survival time of a patient having aggressive bladder cancer after surgery, and beneficially choosing the more appropriate strategies of treatments.

According to the embodiments of the present invention, the method, the kit, the computer-implemented method and the system for in vitro predicting survival time of an individual with bladder cancer after surgery from an individual's biological sample, which beneficially detect expression levels of target gene combination of in vitro aggressive bladder cancer specimen of an individual, respectively compare those expression levels with the reference expression levels of the target gene combination of a reference database, the results can be converted to a risk score sum, thereby increasing the predicting accuracy of an averaged survival time of an individual having aggressive bladder cancer after surgery.

Although the present invention has been described in considerable detail with reference to certain embodiments thereof, other embodiments are possible. Therefore, the spirit and scope of the appended claims should not be limited to the description of the embodiments contained herein.

What is claimed is:

1. A method for predicting survival time of an individual with bladder cancer after surgery from an individual's biological sample, comprising:
establishing a reference data bank, wherein the reference data bank comprises reference expression levels of a target gene combination originated from at least one in vitro normal bladder specimen, the target gene combination comprises at least two of PPT2, ARMH4, P4HB, SLC1A6 and ARID3A, a fragment, a homologue, a variant and a derivative thereof;
providing a biological specimen, wherein the biological specimen is originated from in vitro aggressive bladder cancer of the individual;
detecting expression levels of the target gene combination of the biological specimen;
comparing one of the expression levels with a corresponding reference expression level of the target gene combination of a reference database respectively, and obtaining a difference and a risk score, wherein a risk score of the one is given as 1 when an absolute value of the difference is equal to or more than a first threshold that is at least 5% of the reference expression level; and
calculating a risk score sum of at least two of the target gene combination of the biological specimen, when the risk score sum is equal to or more than a second threshold that is 1 or 2, the individual having the biological specimen is classified to a high risk group, wherein the high risk group is defined by less 25 months of an averaged survival time after surgery of the individual with 1 or 2 of the second threshold, or by less 10 months of an averaged survival time after surgery of the individual with 3 of the second threshold.

2. The method of claim 1, wherein the biological specimen and the in vitro normal bladder specimen comprise ex vivo organs, tissues, cells, body fluid, lymphatic liquid, urine, whole bloods, serum and/or cell culture supernatant.

3. The method of claim 1, wherein each of the reference expression levels is a normalized value.

4. The method of claim 1, wherein the expression levels and the reference expression levels comprise nucleic acid expression levels and/or protein expression levels.

5. A kit for predicting survival time of an individual with bladder cancer after surgery from an individual's biological sample, comprising:
a reaction solution, nucleic acid probes and/or antibodies, wherein the nucleic acid probes and/or antibodies react with a target gene combination of a biological specimen and generate expression levels, the biological specimen is originated from in vitro aggressive bladder cancer of an individual, and the target gene combination is selected from at least two of the group consisting of PPT2, ARMH4, P4HB, SLC1A6 and ARID3A, a fragment, a homologue, a variant and a derivative thereof.

6. The kit of claim 5, wherein the biological specimen comprises ex vivo organs, tissues, cells, body fluid, lymphatic liquid, urine, whole bloods, serum and/or cell culture supernatant.

7. A computer-implemented method for predicting survival time of an individual with bladder cancer after surgery from an individual's biological sample, comprising executing steps as follow:
storing a reference data bank comprising reference expression levels of a target gene combination originated from at least one in vitro normal bladder specimen in a system, wherein the target gene combination comprises at least two of PPT2, ARMH4, P4HB, SLC1A6 and ARID3A, a fragment, a homologue, a variant and a derivative thereof;
detecting expression levels of the target gene combination of a biological specimen originated from in vitro aggressive bladder cancer of the individual;
comparing one of the expression levels of the biological specimen with a corresponding reference expression level of the target gene combination of a reference database respectively, for obtaining a difference and a risk score, wherein a risk score of the one is given as 1 when an absolute value of the difference is equal to or more than a first threshold that is more than at least 5% of the reference expression level;
calculating a risk score sum of at least two of the target gene combination of the biological specimen; and
identifying the individual from the risk score sum, when the risk score sum is equal to or greater than a second threshold that is 1 or 2, the individual having the biological specimen is classified to a high risk group, wherein the high risk group is defined by less 25 months of an averaged survival time after surgery of the individual with 1 or 2 of the second threshold, or by less 10 months of an averaged survival time after surgery of the individual with 3 of the second threshold, and
wherein a program has instructions for executing the step of storing the reference data bank, the step of comparing the one of the expression levels with the corresponding reference expression level of the target gene combination, the step of calculating the risk score sum of the at least two of the target gene combination of the biological specimen, and the step of identifying the individual from the risk score sum, for implementing the method on a system.

8. The computer-implemented method of claim 7, wherein the biological specimen and the in vitro normal bladder specimen comprise ex vivo organs, tissues, cells, body fluid, lymphatic liquid, urine, whole bloods, serum and/or cell culture supernatant.

9. The computer-implemented method of claim 7, wherein each of the reference expression levels is a normalized value.

10. The computer-implemented method of claim 7, wherein the expression levels and the reference expression levels comprise nucleic acid expression levels and/or a protein expression levels.

11. The computer-implemented method of claim 7, wherein the program comprises instructions for implementing the method on the system.

12. The computer-implemented method of claim 7, wherein the system comprises:
a detecting module comprising a detecting device, a reaction solution, nucleic acid probes and/or antibodies, wherein the nucleic acid probes and/or antibodies react with a target gene combination of a biological specimen and generate expression levels, and the detecting device detects the expression levels, the biological specimen is originated from in vitro aggressive bladder cancer of an individual, the target gene combination is selected from the group consisting of at least two of the group consisting of PPT2, ARMH4, P4HB, SLC1A6 and ARID3A, a fragment, a homologue, a variant and a derivative thereof;
a processing module coupled to the detecting module, for receiving the expression levels from the detecting module and comparing one of the expression levels with a corresponding reference expression level of the target gene combination of a reference database respectively, thereby obtaining a difference and a risk score; and for calculating a risk score sum of at least two of the target gene combination of the biological specimen, wherein the risk score of the one is given as 1 when an absolute value of the difference is equal to or more than a first threshold that is more than at least 5% of the reference expression level;
an identifying module coupled to the processing module, for identifying the individual from the risk score sum, wherein when the risk score sum is equal to or greater than a second threshold that is 1 or 2, the individual having the biological specimen is classified to a high risk group, the high risk group is defined by less 25 months of an averaged survival time after surgery of the individual with 1 or 2 of the second threshold, or by less 10 months of an averaged survival time after surgery of the individual with 3 of the second threshold; and
a controlling module coupled to the detecting module, the processing module and the identifying module, and
wherein the system executes a program that includes instructions, thereby activating the detecting module, the processing module and the identifying module.

13. The computer-implemented method of claim 12, further comprising:
a pretreating module coupling to the detecting module, for providing a nucleic acid sample and/or a protein sample of the biological specimen.

14. The computer-implemented method of claim 12, wherein the reference expression level is originated from at least one in vitro normal bladder sample.
